(19) United States
(12) Patent Application Publication
(10) Pub. No.: US 2025/0295879 A1
(43) Pub. Date: Sep. 25, 2025

(54) ENDOBRONCHIAL BLOCKER

(71) Applicants: National Taiwan University Hospital Hsin-Chu Branch, Zhubei City (TW); National Taiwan University, Taipei (TW)

(72) Inventor: CHUN-YU WU, Zhubei City (TW)

(21) Appl. No.: 19/079,976

(22) Filed: Mar. 14, 2025

Related U.S. Application Data
(60) Provisional application No. 63/567,771, filed on Mar. 20, 2024.

(51) Int. Cl. A61M 16/04

(52) U.S. Cl. CPC A61M 16/0406; A61M 16/045

(57) ABSTRACT

An endobronchial blocker includes an operation box, an extension tube, a ballon, an inflation module, and an elbow module. The extension tube is connected to the operation box. The extension tube has a first passageway and at least one second passageway therein. The ballon is disposed on one end of the extension tube and is in communication with the first passageway. The ballon is in a deflated state during movement. The inflation module is connected to the first passage for supplying a gas to the ballon. The elbow module is assembled to the operation box and includes a pulling means and a control handle. When in use, the control handle is operated to move the pulling means, enabling the extension tube to bend or straighten, thereby reducing the risk of surgical interruption due to poor quality of the left/right lung respiration.

### BACKGROUND OF THE INVENTION

### 1. Field of the Invention

The present invention relates to a medical auxiliary device, and more particularly to an endobronchial blocker, which utilizes integrated innovation to solve the clinical problems by providing a ballon image monitoring module and an elbow module to enhance its applicability, so as to achieve precise angle adjustments for the ballon to reach a desired position during intubation and to monitor the blocking state of the ballon in real time, thus reducing the risk of surgical interruption greatly.

### 2. Description of the Related Art

A conventional endobronchial blocker on the market is designed based on the tracheal tube. However, because the front end of the endobronchial blocker has an inflatable ballon as a means to be secured to the wall of the bronchial tube, it needs to be placed inside the tracheal tube to reach a desired position of the bronchial tube. After the ballon reaches the desired position, it is inflated to be secured on the inner wall of the bronchial tube. However, during thoracic surgery, the ballon of the endobronchial blocker may be displaced due to surgical tension, resulting in the interruption of the left/right lung respiration and the need to readjust the position of the endobronchial blocker. Therefore, it is necessary for the endobronchial blocker to cooperate with an image flexible tube when in use. Besides, the front end of the endobronchial blocker has a curved section to guide the front end to be inserted in the patient's bronchial tube, so that the medical staff can rotate the endobronchial blocker to change its direction. However, the conventional endobronchial blocker has two major drawbacks. (1) The endobronchial blocker itself does not have an image flexible tube. Additional bronchoscopy is required to confirm the position of the intubation and to reposition it case of displacement during surgery. However, the narrow space inside the endotracheal tube often causes inconvenience in operation. According to clinical statistics, more than 90% of physicians believe that they have this problem. (2) The conventional endobronchial blocker is made of plastic material, which cannot be rotated flexibly. Intraoperative readjustments often cause surgical interruptions and other issues. Therefore, there is an urgent need for the medical profession to address these shortcomings.

### SUMMARY OF THE INVENTION

The primary objective of the present invention is to provide an endobronchial blocker, comprising an operation box, an extension tube, a ballon, an inflation module, and an elbow module. The operation box is combined with the extension tube and the elbow module to control the movement and direction of the ballon during placement. A ballon image monitoring module is configured for internal illumination and image capture during intubation, so as to confirm the correct positions of the extension tube and the ballon to be inserted in a patient's bronchial tube. Then, the inflation module inflates the ballon so that the ballon is secured at a predetermined position to block off the patient's bronchial tube, and the blocking state of the ballon is monitored in real time through the ballon image monitoring module.

In order to achieve the foregoing objective, the present invention provides an endobronchial blocker. When in use, the endobronchial blocker is inserted in a patient's bronchial tube for medical treatment. The endobronchial blocker comprises an operation box, an extension tube, a ballon, an inflation module, and an elbow module. The operation box has a first accommodation room and a second accommodation room therein. A communication hole is formed between the first accommodation room and the second accommodation room. The first accommodation room is formed with an operation hole relative to a top of the operation box and a connection hole relative to a bottom of the operation box. The extension tube is connected to the second accommodation room. The extension tube has a first passageway and at least one second passageway therein. The extension tube further has a first end portion, a second end portion, and a mounting portion on an outer surface of the extension tube close to the first end portion. The ballon is disposed on a surface of the first end portion so that the ballon is in communication with the first passageway. The ballon is in a deflated state during movement. The inflation module has an inflation tube connected to the first passage at the second end portion for supplying a gas to the ballon to be inflated. The elbow module includes a pulling means and a control handle. One end of the pulling means is inserted into the extension tube and connected to the first end portion, and another end of the pulling means is linked to the control handle. The control handle is operative to bend or straighten the extension tube.

Preferably, the endobronchial blocker further comprises a ballon image monitoring module. When in use, the ballon image monitoring module and the elbow module control the extension tube to reach a predetermined position of the patient's bronchial tube, and the inflation module inflates the ballon so that the ballon is secured at the predetermined position to block off the patient's bronchial tube, and a blocking state of the ballon is monitored in real time.

### BRIEF DESCRIPTION OF THE DRAWINGS

FIG. 1 is an exploded view of a preferred embodiment of the present invention;

FIG. 2 is a perspective view of the preferred embodiment of the present invention;

FIG. 3 is a schematic view of the preferred embodiment of the present invention in a first operation state when in use;

FIG. 4 is a structural schematic view of the preferred embodiment of the present invention corresponding to the first operation state;

FIG. 5 is a schematic view of the preferred embodiment of the present invention in a second operation state when in use;

FIG. 6 is a structural schematic view of the preferred embodiment of the present invention corresponding to the second operation state;

FIG. 7 is a schematic view of the preferred embodiment of the present invention in a third operation state when in use;

FIG. 8 is a structural schematic view of the preferred embodiment of the present invention corresponding to the third operation state;

FIG. 9 is a schematic view of the preferred embodiment of the present invention, illustrating that the blocking state of the ballon is monitored by the ballon image monitoring module;

FIG. 10 is another schematic view of the preferred embodiment of the present invention, illustrating that the blocking state of the ballon is monitored by the ballon image monitoring module; and

FIG. 11 is a further schematic view of the preferred embodiment of the present invention, illustrating that the blocking state of the ballon is monitored by the ballon image monitoring module.

### DETAILED DESCRIPTION OF THE PREFERRED EMBODIMENTS

The above and other objects, features and advantages of this disclosure will become apparent from the following detailed description taken with the accompanying drawings.

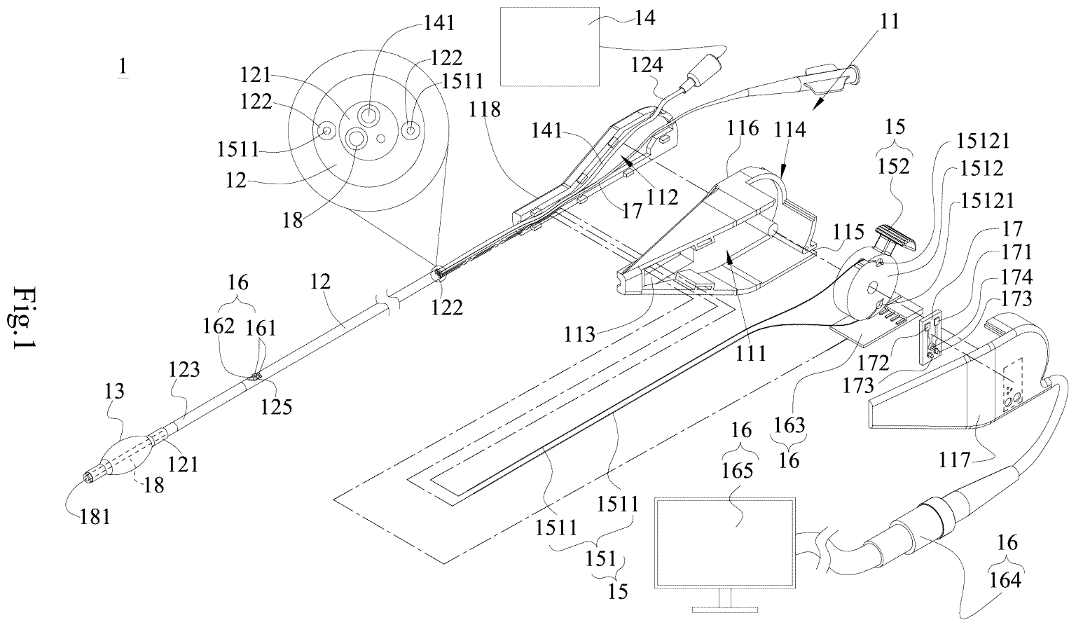

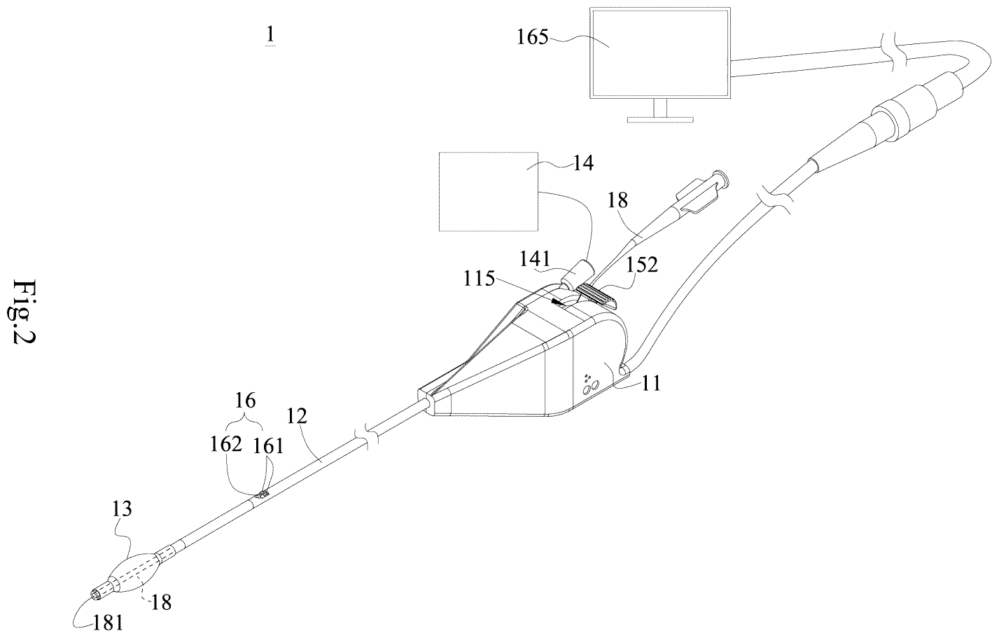

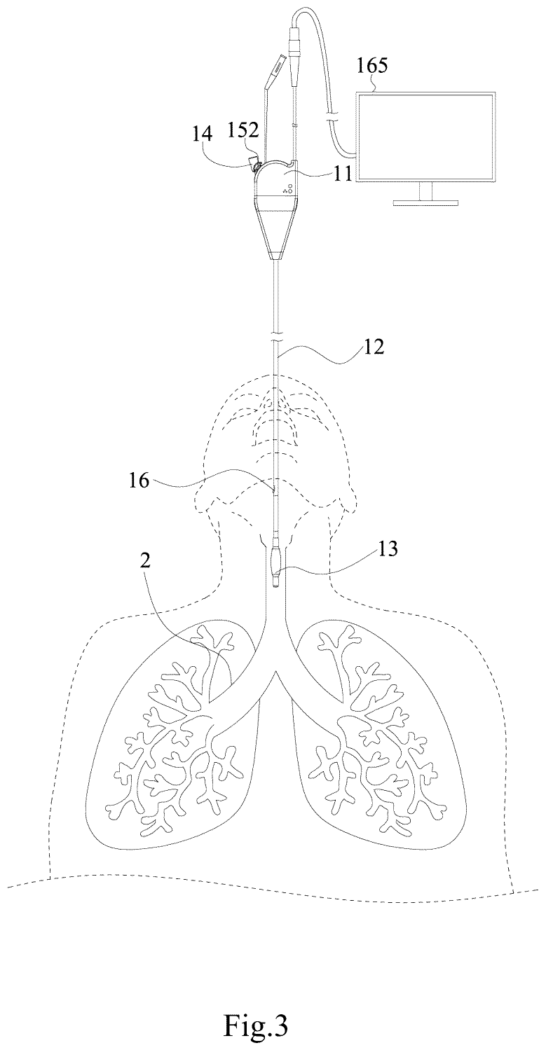

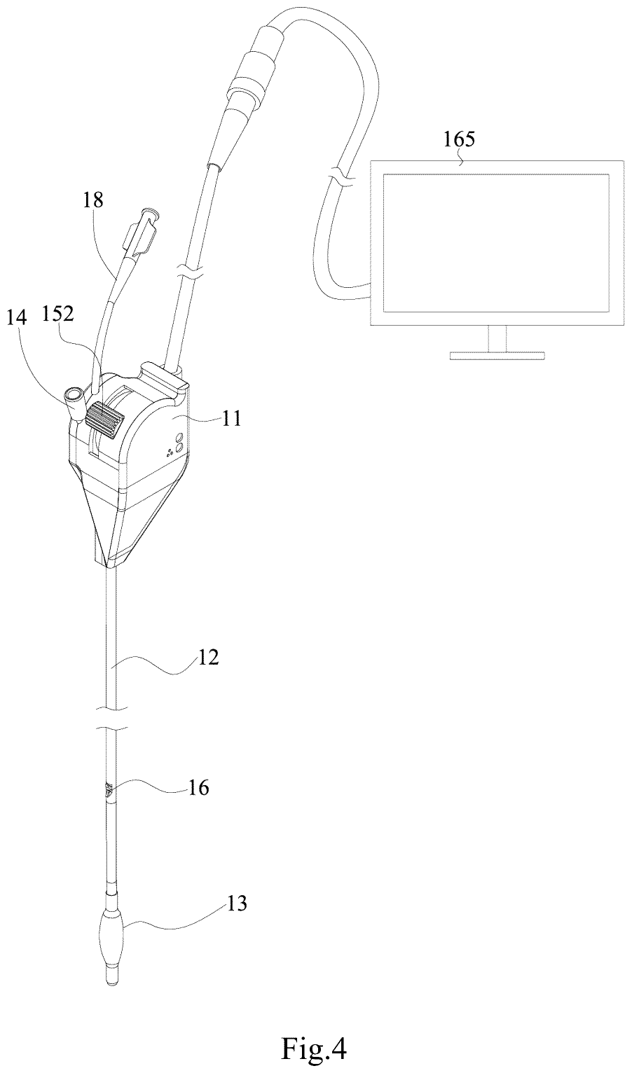

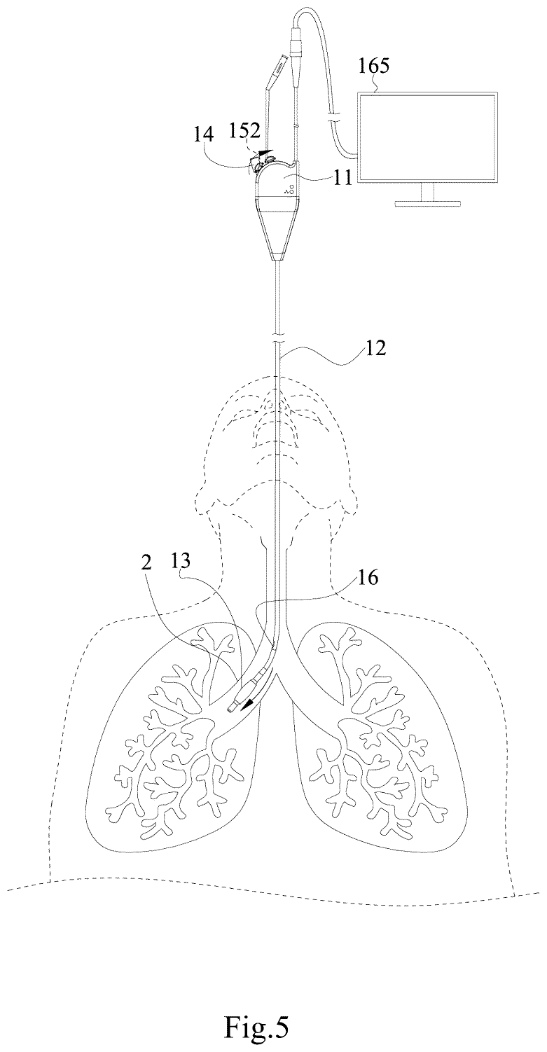

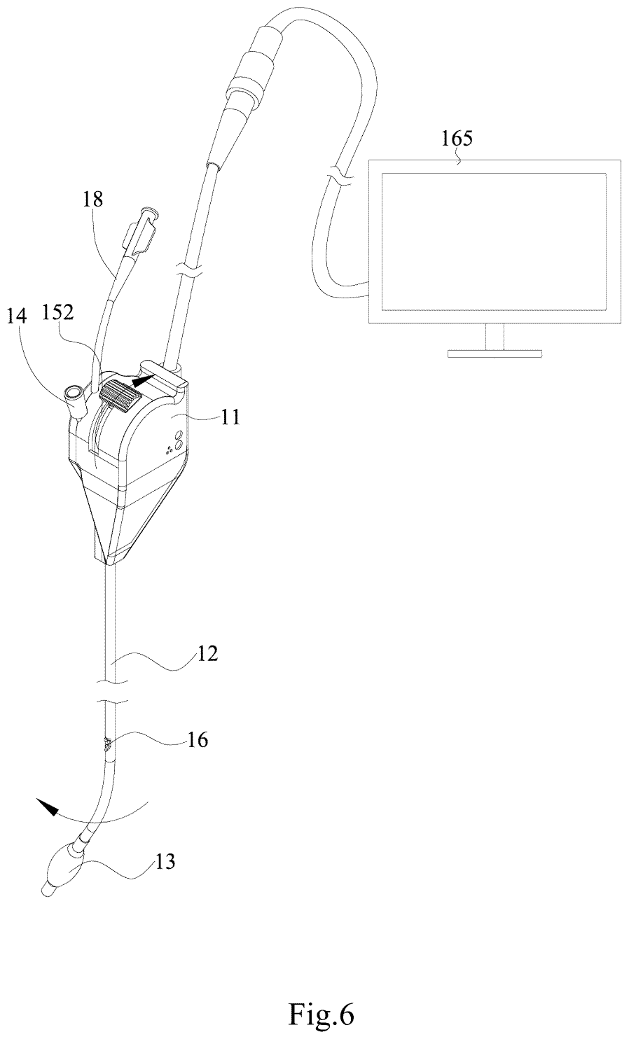

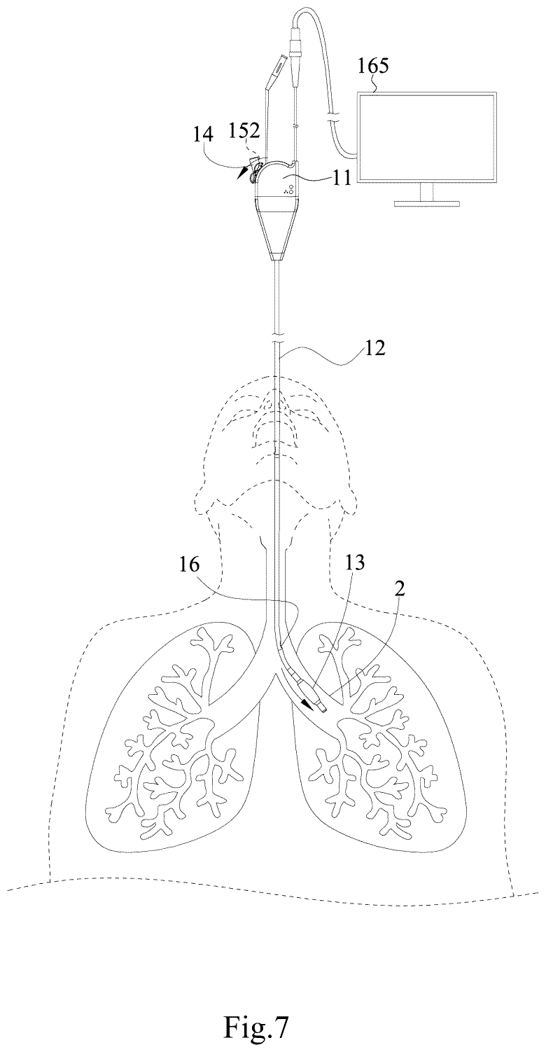

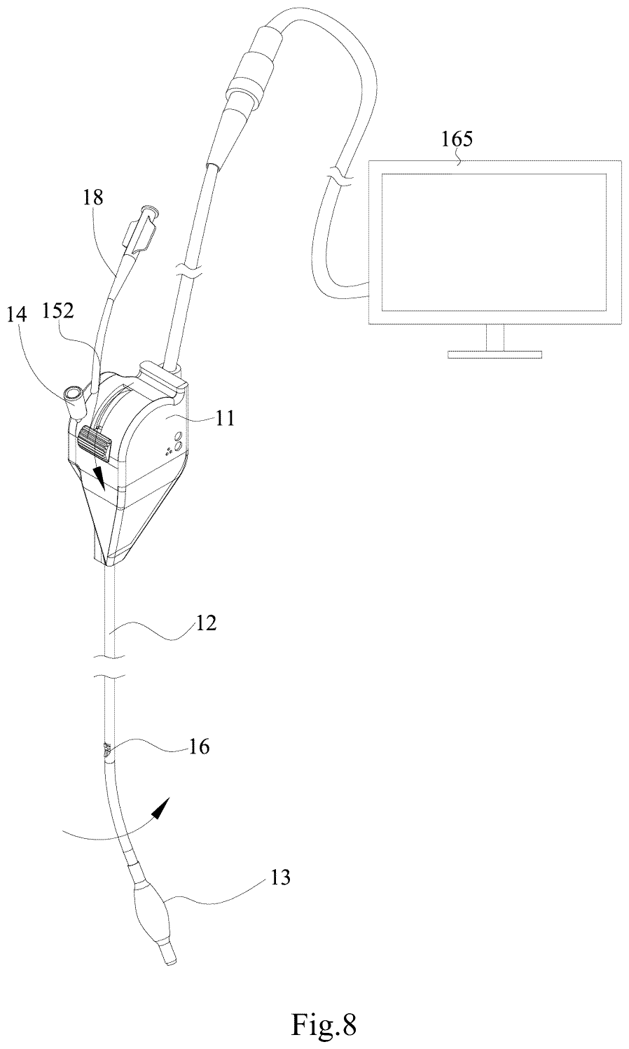

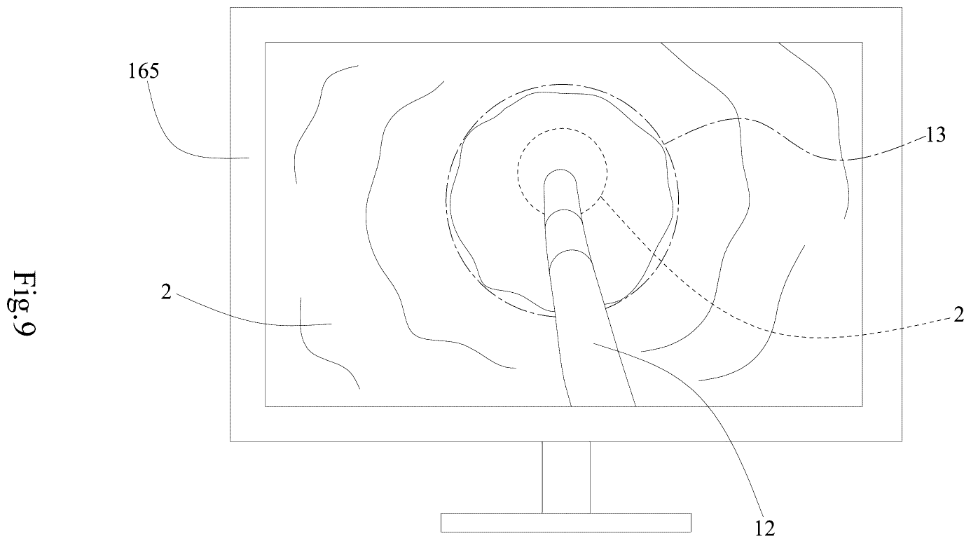

FIG. 1 and FIG. 2 are an exploded view and a perspective view of a preferred embodiment of the present invention. FIGS. 3 to 8 are state schematic views and structural schematic view of the preferred embodiment of the present invention in different operations. FIGS. 9 to 11 are schematic views of the preferred embodiment of the present invention in different blocking states. As shown in the figures, the present invention discloses an endobronchial blocker 1. The endobronchial blocker 1 comprises an operation box 11, an extension tube 12, a ballon 13, an inflation module 14, an elbow module 15, and a ballon image monitoring module 16. When in use, the endobronchial blocker 1 is inserted in a patient's bronchial tube 2 for medical treatment.

The operation box 11 has a first accommodation room 111 and a second accommodation room 112 therein. A communication hole 113 is formed between the first accommodation room 111 and the second accommodation room 112. The first accommodation room 111 is formed with an operation hole 114 relative to the top of the operation box 11 and a connection hole 115 relative to the bottom of the operation box 11. The operation box 11 of the present invention includes a body 116, a first side cover 117, and a second side cover 118. The first side cover 117 is assembled to one side of the body 116 to form the first accommodation room 111, and the second side cover 118 is assembled to another side of the body 116 to form the second accommodation room 112.

The extension tube 12 is connected to the second accommodation room 112. The extension tube 12 has a first passageway 121 and at least one second passageway 122 therein. The extension tube 12 further has a first end portion 123, a second end portion 124, and a mounting portion 125 on the outer surface of the extension tube 12 close to the first end portion 123. The extension tube 12 of the present invention extends out of the operation box 11 from the second accommodation room 112. In a specific implementation, the extension tube 12 has a first passageway 121 and a pair of second passageways 122. The pair of second passageways 122 are disposed at two opposite sides of the first passageway 121. When installed, the pair of second passageways 122 are arranged in a horizontal direction when the operation box 11 is in operation.

The ballon 13 is mounted on the surface of the first end portion 123 so that the ballon 13 is in communication with the first passageway 121. The ballon 13 is in a deflated state during movement.

The inflation module 14 has an inflation tube 141 connected to the first passage 121 at the second end portion 124 for supplying a gas to the ballon 13 to be inflated.

The elbow module 15 includes a pulling means 151 and a control handle 152. One end of the pulling means 151 is inserted into the extension tube 12 and connected to the first end portion 123, and the other end of the pulling means 151 is linked to the control handle 152. The extension tube 12 can be bent or straightened by operating the control handle 152. In a specific implementation, the elbow module 15 includes two pull ropes 1511, a control handle 152, and a rotary disk 1512. The pull ropes 1511 are inserted into the second passageways 122, respectively. The pull ropes 1511 each have one end fixed to the first end portion 123 and another end passing through the communication hole 113 and connected to a corresponding one of two mounting holes 15121 of the rotary disk 1512. The control handle 152 is linked to the rotary disk 1512. By operating the control handle 152, one pull rope 1511 is extended and the other pull rope 1511 is retracted, enabling the extension tube 12 to be bent or straightened. The two mounting holes 15121 are respectively disposed at two diagonal positions of the rotary disk 1512. The two diagonal positions are 180 degrees. Each diagonal position is at 90 degrees to the position where the corresponding pull rope 1511 enters the rotary disk 1512. In addition, the operation hole 114 is linearly configured. The control handle 152 is operated along the extension direction of the operation hole 114.

The ballon image monitoring module 16 includes a lighting element 161, an image capturing element 162, and a control signal element 163. The lighting element 161 and the image capturing element 162 are electrically connected to the signal control element 163. The lighting element 161 and the image capturing element 162 are spaced apart from each other and disposed on the mounting portion 125. The control signal element 163 is mounted inside the operation box 11, corresponding in position to the connection hole 115. In this embodiment, the ballon image monitoring module 16 includes two spaced lighting elements 161. The two lighting elements 161 are light-emitting diodes. The image capturing element 162 is a complementary metal oxide semiconductor. In order to facilitate the connection for monitoring and control, the endobronchial blocker 1 further has a signal port 164 which is electrically connected to the signal control element 163. The signal port 164 is disposed at the connection hole 115 and configured for connecting a monitor 165.

To enhance the safety and convenience of the operation, the endobronchial blocker 1 further comprises an alarm module 17. The alarm module 17 is disposed on the surface of the operation box 11 and electrically connected to the image capturing element 162. The alarm module 17 includes an image analyzing element 171, an image database 172, at least one light indicator 173, and a buzzer 174. The image analyzing element 171 analyzes the image obtained by the image capturing element 162 and compares the contour and the circumference of the ballon 13 in the image database 172. When the exposed contour of the ballon 13 is greater one-sixth to one-twelfth of the circumference of the ballon 13, an abnormality is detected and an alarm is given by the light indicator 173 and the buzzer 174.

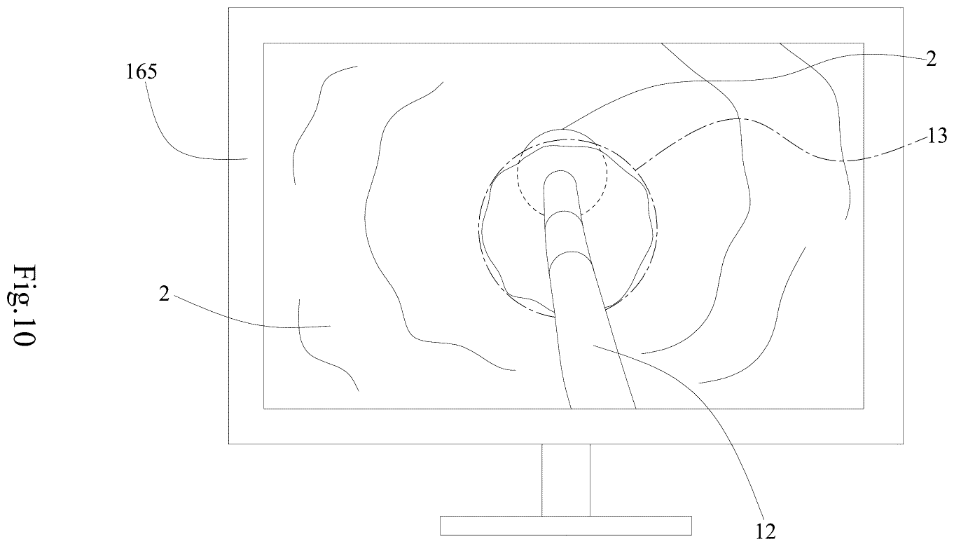

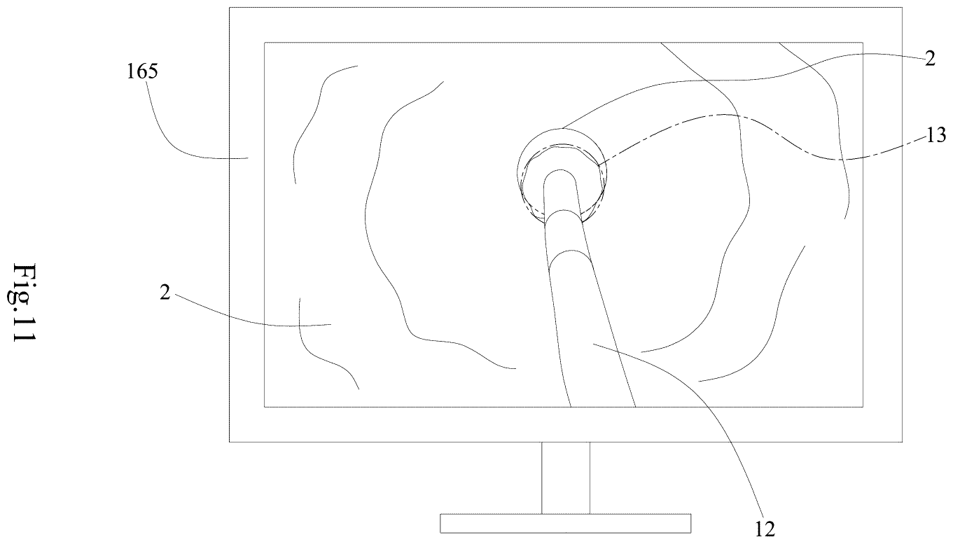

Furthermore, the endobronchial blocker 1 provided by the present invention further comprises a gas supply tube 18 to facilitate surgery. The gas supply tube 18 passes through the first passageway 121 inside the extension tube 12 along the second accommodation room 112. One end of the gas supply tube 18 is located at the first end portion 123 to form a gas supply port 181. The gas supply port 181 is spaced apart from the ballon 13 at a distance from a position where the ballon 13 is to block off the patient's bronchial tube for gas supply. FIGS. 9 to 11 are schematic views of the present invention used to block off the patient's bronchial tube. FIG. 9 illustrates a fully blocked state. When the image analyzing element 171 detects that the tissues on the surface of the bronchial tube 2 have been expanded by the ballon 13 to form a tight seal, the exposed contour of the ballon 13 is less than 1/24 to 0 of the circumference of the ballon 13, which indicates that it has been blocked by the tissues on the surface of the bronchial tube 2 so that the contour of the ballon 13 is nearly invisible. The contour of the ballon 13 in FIG. 9 is indicated by a dotted line. The alarm module 17 does not give any alarm. FIG. 10 illustrates a non-blocked state. When the image analyzing element 171 detects that the ballon 13 is slightly displaced to the tissues on the surface of the bronchial tube 2 and the exposed contour of the ballon 13 is more than one twelfth of the circumference of the ballon 13, the alarm module 17 will light up one light indicator 173 and the buzzer 174 will emit a low-frequency alarm to alert the medical staff to make adjustments. FIG. 11 illustrates that the ballon 13 is greatly deflated or displaced. When the image analyzing element 171 detects that the exposed contour of the ballon 13 is more than one sixth of the circumference of the ballon 13 and the bronchial tube 2 is not fully blocked, the alarm module 17 will light up two light indicators 173 and the buzzer 174 will emit a high-frequency alarm to alert the medical staff that the ballon 13 is greatly displaced or deflated or the inflation device is damaged so the surgery cannot be performed.

Accordingly, the endobronchial blocker 1 utilizes the extension tube 11 in combination with the ballon 13 and the ballon image monitoring module 16 for internal illumination and image capture during intubation. The ballon image monitoring module 16 is operated through the operation box 12. The ballon 13 is inflated by the inflation module 14. The ballon image monitoring module 16 is configured to confirm whether the movement and direction of the extension tube 11 are correct. The elbow module 15 controls the correct position of the extension tube 11 to be inserted in the patient's bronchial tube 2. The inflation module 14 inflates the ballon 13 so that the ballon 13 is secured at a predetermined position to block off the patient's bronchial tube 2, and the blocking state of the ballon 13 is monitored in real time to avoid problems and prevent surgical interruptions, thereby enhancing the accuracy, stability, and safety of intubation surgery.

What is claimed is:

1. An endobronchial blocker, comprising:
an operation box, having a first accommodation room and a second accommodation room therein, a communication hole being formed between the first accommodation room and the second accommodation room, the first accommodation room being formed with an operation hole relative to a top of the operation box and a connection hole relative to a bottom of the operation box;
an extension tube, connected to the second accommodation room, the extension tube having a first passageway and at least one second passageway therein, the extension tube further having a first end portion, a second end portion, and a mounting portion on an outer surface of the extension tube close to the first end portion;
a ballon, disposed on a surface of the first end portion so that the ballon is in communication with the first passageway, the ballon being in a deflated state during movement;
an inflation module, having an inflation tube connected to the first passage at the second end portion for supplying a gas to the ballon to be inflated; and
an elbow module, including a pulling means and a control handle, one end of the pulling means being inserted into the extension tube and connected to the first end portion, another end of the pulling means being linked to the control handle, the control handle being operative to bend or straighten the extension tube.

2. The endobronchial blocker as claimed in claim 1, further comprising a ballon image monitoring module, wherein the ballon image monitoring module includes a lighting element, an image capturing element and a control signal element, the lighting element and the image capturing element are electrically connected to the signal control element, the lighting element and the image capturing element are spaced apart from each other and disposed on the mounting portion, the control signal element is disposed inside the operation box, corresponding in position to the connection hole; when in use, the ballon image monitoring module and the elbow module control the extension tube to reach a predetermined position of a patient's bronchial tube, and the inflation module inflates the ballon so that the ballon is secured at the predetermined position to block off the patient's bronchial tube, and a blocking state of the ballon is monitored in real time.

3. The endobronchial blocker as claimed in claim 2, further comprising an alarm module, wherein the alarm module is disposed on a surface of the operation box and electrically connected to the image capturing element, the alarm module includes an image analyzing element, an image database, at least one light indicator and a buzzer, the image analyzing element analyzes an image obtained by the image capturing element and compares the image with the image database, when an abnormality is detected, an alarm is given by the light indicator and the buzzer.

4. The endobronchial blocker as claimed in claim 3, wherein the elbow module includes two pull ropes, a control handle and a rotary disk, each of the pull ropes is inserted in the corresponding second passageway, the pull ropes each have one end fixed to the first end portion and another end passing through the communication hole and connected to a corresponding one of two mounting holes of the rotary disk, the control handle is linked to the rotary disk, by operating the control handle, one of the two pull ropes is extended and the other one of the two pull ropes is retracted, enabling the extension tube to be bent or straightened.

5. The endobronchial blocker as claimed in claim 4, wherein the two mounting holes are respectively disposed at two diagonal positions of the rotary disk, the two diagonal positions are 180 degrees, and each of the two diagonal positions is at 90 degrees to a position where a corresponding one of the pull ropes enters the rotary disk.

6. The endobronchial blocker as claimed in claim 5, wherein the operation box includes a body, a first side cover and a second side cover, the first side cover is assembled to one side of the body to form the first accommodation room, and the second side cover is assembled to another side of the body to form the second accommodation room.

7. The endobronchial blocker as claimed in claim 6, wherein the extension tube extends out of the operation box from the second accommodation room, the at least one second passageway includes a pair of second passageways, the pair of second passageways are disposed at two opposite sides of the first passageway, when installed, the pair of second passageways are arranged in a horizontal direction when the operation box is in operation.

8. The endobronchial blocker as claimed in claim 7, further comprising a gas supply tube, wherein the gas supply tube is inserted in the extension tube along the second accommodation room, one end of the gas supply tube is located at the first end portion to form a gas supply port, and the gas supply port is spaced apart from the ballon at a distance from a position where the ballon is to block off the patient's bronchial tube for gas supply.

9. The endobronchial blocker as claimed in claim 2, wherein the ballon image monitoring module includes two spaced lighting elements, the two lighting elements are light-emitting diodes, and the image capturing element is a complementary metal oxide semiconductor.

10. The endobronchial blocker as claimed in claim 2, wherein the operation hole is linearly configured, and the control handle is operated along an extension direction of the operation hole.

11. The endobronchial blocker as claimed in claim 10, further comprising a signal port electrically connected to the signal control element, wherein the signal port is disposed at the connection hole and configured for connecting a monitor.
